(19) United States
(12) Patent Application Publication
(10) Pub. No.: US 2007/0010522 A1
(43) Pub. Date: Jan. 11, 2007

(54) Triazolo[1,5-c]pyrimidines and pyrazolo[1,5-c]pyrimidines useful as a2a adenosine receptor antagonists

(76) Inventors: Chi Vu, Arlington, MA (US); Russell C. Petter, Stow, MA (US); Gnanasambandam Kumaravel, Westford, MA (US)

Correspondence Address: Biogen Idec Inc;Patent and Trademark Coordinator, 14 Cambridge Center, Cambridge, MA, 02142, US

(21) Appl. No.: 10/552,302

(22) PCT Filed: Apr. 9, 2004
(86) PCT No.: PCT/US04/11008

§ 371 Date: Jul. 26, 2006

Related U.S. Application Data
(60) Provisional application No. 60/461,355, filed on Apr. 9, 2003.

(51) Int. Cl. A61K 31/519; C07D 487/04

(52) U.S. Cl. 514/252.16; 514/259.3; 514/259.31; 544/280; 544/281

(57) ABSTRACT

The invention is based on the discovery that compounds of formula (I) possess unexpectedly high affinity for the A2a adenosine receptor, and can be useful as antagonists thereof for preventing and/or treating numerous diseases, including Parkinson's disease. In one embodiment, the invention features a compound of formula (I).

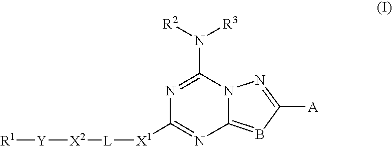

### BACKGROUND OF THE INVENTION

Adenosine is a ubiquitous biochemical messenger. Adenosine binds to and activates certain seven transmembrane-spanning G-protein coupled receptors, eliciting a variety of physiological responses. Adenosine receptors are divided into four known subtypes (i.e. A₁, A₂ₐ, A_{2b}, and A₃). These receptor subtypes mediate different and sometimes opposing effects. In general, activation of the adenosine A₂ₐ or A_{2b} receptor leads to an increase in cellular cAMP levels, while activation of the adenosine A₁ or A₃ receptor leads to a decrease in cellular cAMP levels. A₂ₐ adenosine receptors are abundant in the basal ganglia, a region of the brain associated with the pathphysiology of Parkinson's disease. For reviews concerning A adenosine receptors, see, e.g., Moreau et al., Brain Research Reviews 31:65-82 (1999) and Svenningsson et al., Progress in Neurobiology 59:355-396(1999). For a discussion of the role and regulation of adenosine in the central nervous system, see, e.g., Dunwiddie et al., Ann. Rev. Neuroscience 24:31-55 (2001).

### SUMMARY OF THE INVENTION

The invention is based on the discovery that compounds of formula (I) are unexpectedly potent antagonists of the A₂ₐ subtype of adenosine receptors. Many compounds of formula (I) also selectively inhibit the A₂ₐ adenosine receptors. Adenosine antagonists of the present invention are useful in the prevention and/or treatment of various diseases and disorders related to modulation of A₂ₐ adenosine receptor signaling pathways. Such a disease or disorder can be, e.g., neurodegenerative diseases such as Parkinson's disease and Parkinson's-like syndromes such as progressive supranuclear palsy and multiple system atrophy, senile dementia such as Alzheimer's disease, depression, AIDS encephalopathy, multiple sclerosis, amyotrophic lateral sclerosis, migraine, attention deficit disorder, narcolepsy, sleep apnea or other disorders that cause excessive daytime sleepiness, Huntington's disease, cerebral ischemia, brain trauma, hepatic fibrosis, cirrhosis, and fatty liver.

In one aspect, the invention features compounds of formula (I):

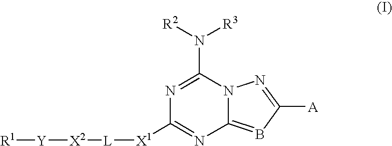

A can be aryl or heteroaryl. B can be N or CR². Each of R² and R³, independently, can be hydrogen, alkyl, cycloalkyl, cycloalkenyl, aryl, aralkyl, heterocycloalkyl, heterocycloalkenyl, heteroaryl, or heteroaralkyl. Each of X¹ and X², independently, can be C₁₋₆ alkylene, C₂₋₆ alkenylene, C₂₋₆ alkynylene, or a bond. Y can be —C(R²)(R³)—, —O—, —S—, —SO—, —SO₂—, —CO—, —CO₂—, or a bond. R¹ can be alkyl, alkenyl, alkynyl, cycloalkyl, cycloalkylalkyl, cycloalkenyl, cycloalkenylalkyl, aryl, aralkyl, heterocyclyl, or heterocyclylalkyl. L can be a bond or a linker selected from the group consisting of: 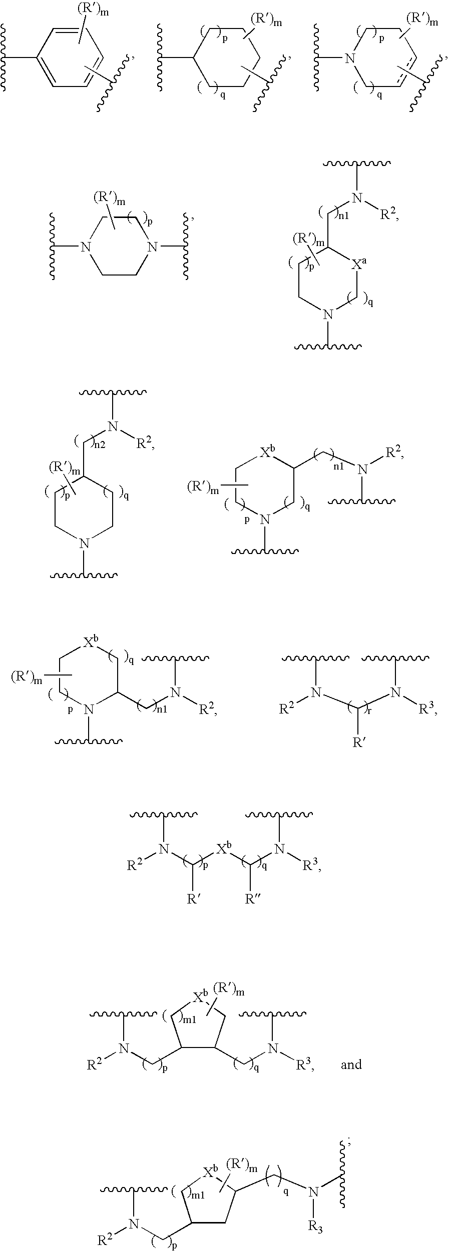
wherein each of R′ and R″, independently, can be hydrogen, alkyl, alkenyl, alkynyl, alkoxy, acyl, halo, hydroxy, amino, nitro, oxo, thioxo, cyano, guanadino, amidino, carboxy, sulfo, sulfoxy, mercapto, alkylsulfanyl, alkylsulfinyl, alkylsulfonyl, aminocarbonyl, alkylcarbonylamino, alkylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, heterocycloalkyl, heterocycloalkyloxy, heterocycloalkylsulfanyl, aryl, aryloxy, arylsulfanyl, aroyl, heteroaryl, heteroaryloxy, heteroarylsulfanyl, or heteroaroyl (note that two adjacent R′ groups can join together to form a 4- to 8-membered optionally substituted cyclic moiety); X^{a} can be —C(R²)(R³)—, —S—, —SO—, or —SO₂—; X^{b} can be —C(R²)(R³)—, —NR²—, —O—, —S—, —SO—, or —SO₂—; each of p, q, m, and m1, independently, can be 0-3; r can be 1 or 2; n1 can be 0-6; and n2 can be 2-6. Note that (1) when L is 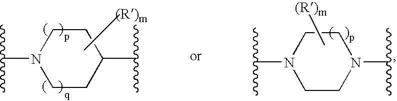
then X¹ is C₁₋₆ alkylene, C₂₋₆ alkenylene, or C₂₋₆ alkynylene; (2) when L is 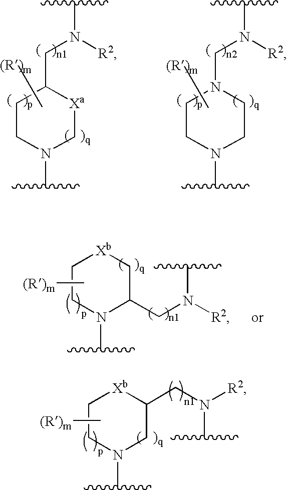
then R¹ is aryl or heteroaryl; and (3) when L is a bond, then X¹ is an alkynylene.

In one embodiment, X¹ can be C₂₋₆ alkynylene.

In one embodiment, L can be 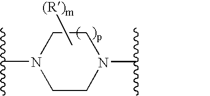
or a bond.

In one embodiment, X² can be C₁₋₄ alkylene or a bond.

In one embodiment, Y can be a bond.

In one embodiment, each of R² and R³, independently, can be hydrogen or alkyl.

In one embodiment, R¹ can be alkyl, cycloalkyl, aryl, heterocycloalkyl, or heteroaryl; each of the alkyl, cycloalkyl, aryl, heterocycloalkyl, and heteroaryl is optionally substituted with alkyl, halo, hydroxy, or phenyl.

In one embodiment, X¹ can be C₂₋₆ alkynylene; L can be 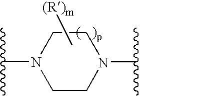
or a bond; X² can be C₁₋₄ alkylene or a bond; Y can be a bond; each of R² and R³, independently, can be hydrogen or alkyl; R¹ can be alkyl, cycloalkyl, aryl, heterocycloalkyl, or heteroaryl, each of which being optionally substituted with alkyl, halo, hydroxy, or phenyl; A can be heteroaryl; and B can be N.

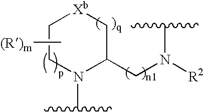

In one embodiment, L can be 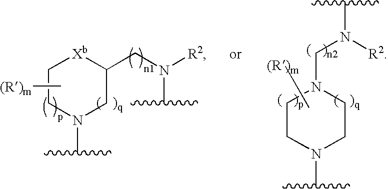
For example, X^{b} can be —C(R²)(R³)— or —NR²—(e.g., X^{b} can be —C(R²)(R³)— such as —CH₂—); p can be 0-1; q can be 1; n1 can be 1-4 and n2 can be 2-4.

In one embodiment, X¹ can be C₁₋₆ alkylene or a bond.

In one embodiment, X² can be C₁₋₆ alkylene or a bond.

In one embodiment, Y can be —SO₂—, —CO—, —CO₂—, or a bond.

In one embodiment, each of R² and R³, independently, can be hydrogen or alkyl.

In one embodiment, R¹ can be aryl or heteroaryl; each of the aryl and heteroaryl can be substituted with alkyl, halo, hydroxy, or phenyl.

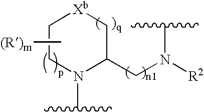

In one embodiment, L can be 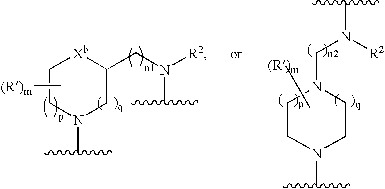
(wherein X^{b} can be —C(R²)(R³)— or —NR²— (e.g., X^{b} can be —C(R²)(R³)— such as —CH₂—); p can be 0-1; q can be 1; n1 can be 1-4 and n2 can be 2-4); each of X¹ and X², independently, can be C₁₋₆ alkylene or a bond, Y can be —SO₂—, —CO—, —CO₂—, or a bond; each of R² and R³, independently, can be hydrogen or alkyl; and R¹ can be aryl or heteroaryl, each of which being optionally substituted with alkyl, halo, hydroxy, or phenyl.

In one embodiment, L can be 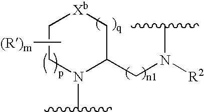
(wherein X^{b} can be —C(R²) (R³)— or —NR²—(e.g., X^{b} can be —C(R²)(R³)— such as —CH₂—); p can be 0-1; q can be 1; n1 can be 1-4 and n2 can be 2-4); X¹ can be a bond; X² can be C₁₋₄ alkylene; Y can be a bond; each of R² and R³, independently, can be hydrogen or alkyl; R¹ can be aryl or heteroaryl, each of which being optionally substituted with alkyl, halo, hydroxy, or phenyl; A can be heteroaryl; and B can be N.

In one embodiment, L can be

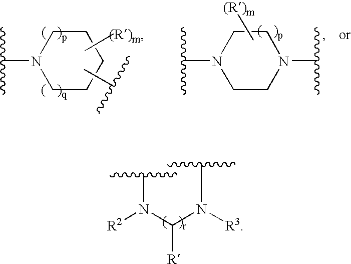

In one embodiment, X¹ can be C₁₋₆ alkylene, C₂₋₆ alkynylene, or a bond.

In one embodiment, X² can be C₁₋₆ alkylene or a bond.

In one embodiment, Y can be —SO₂—, —CO—, —CO₂—, or a bond.

In one embodiment, each of R² and R³, independently, can be hydrogen or alkyl.

In one embodiment, R¹ can be alkyl, cycloalkyl, aryl, heterocycloalkyl, or heteroaryl; each of which can be substituted with alkyl, halo, hydroxy, or phenyl.

In one embodiment, L can be 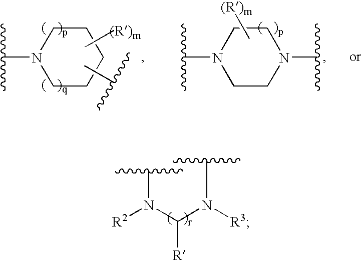
X¹ can be C₁₋₆ alkylene, C₂₋₆ alkynylene, or a bond; X² can be C₁₋₆ alkylene or a bond; Y can be —SO₂—, —CO—, —CO₂—, or a bond; each of R² and R³, independently, can be hydrogen or alkyl; R¹ can be alkyl, cycloalkyl, aryl, heterocycloalkyl, or heteroaryl, each of which being optionally substituted with alkyl, halo, hydroxy, or phenyl; A can be heteroaryl; and B can be N.

Some examples of a compound of formula (I) are shown in Examples 1-32 below.

An N-oxide derivative or a pharmaceutically acceptable salt of each of the compounds of formula (I) is also within the scope of this invention. For example, a nitrogen ring atom of the triazolotriazine or the pyrazolotriazine core ring or a nitrogen-containing heterocyclyl substituent can form an oxide in the presence of a suitable oxidizing agent such as m-chloroperbenzoic acid or H₂O₂.

A compound of formula (I) that is acidic in nature (e.g., having a carboxyl or phenolic hydroxyl group) can form, a pharmaceutically acceptable salt such as a sodium, potassium, calcium, or gold salt. Also within the scope of the invention are salts formed with pharmaceutically acceptable amines such as ammonia, alkyl amines, hydroxyalkylamines, and N-methylglycamine. A compound of formula (I) can be treated with an acid to form acid addition salts. Examples of such an acid include hydrochloric acid, hydrobromic acid, hydroiodic acid, sulfuric acid, methanesulfonic acid, phosphoric acid, p-bromophenyl-sulfonic acid, carbonic acid, succinic acid, citric acid, benzoic acid, oxalic acid, malonic acid, salicylic acid, malic acid, fumaric acid, ascorbic acid, maleic acid, acetic acid, and other mineral and organic acids well known to a skilled person in the art. The acid addition salts can be prepared by treating a compound of formula (I) in its free base form with a sufficient amount of an acid (e.g., hydrochloric acid) to produce an acid addition salt (e.g., a hydrochloride salt). The acid addition salt can be converted back to its free base form by treating the salt with a suitable dilute aqueous basic solution (e.g., sodium hydroxide, sodium bicarbonate, potassium carbonate, or ammonia). Compounds of formula (I) can also be, e.g., in a form of achiral compounds, racemic mixtures, optically active compounds, pure diastereomers, or a mixture of diastereomers.

Compounds of formula (I) exhibit surprisingly high affinity to the A₂ₐ subtype of adenosine receptors, e.g., with Kᵢ values of less than 10 μM under conditions as described in Example 33. Some compounds of formula (I) exhibit Kᵢ values of below 1 μM. Many compounds of formula (I) are selectively inhibitors of the A₂ₐ adenosine receptors (e.g., these compounds inhibit the A₂ₐ adenosine receptors at least 10 times better than the other subtypes of adenosine receptors, e.g., the A₁ adenosine receptors or the A₃ adenosine receptors).

Compounds of formula (I) can also be modified by appending appropriate functionalities to enhance selective biological properties. Such modifications are known in the art and include those that increase biological penetration into a given biological system (e.g., blood, lymphatic system, central nervous system), increase oral availability, increase solubility to allow administration by injection, alter metabolism, and/or alter rate of excretion. Examples of these modifications include, but are not limited to, esterification with polyethylene glycols, derivatization with pivolates or fatty acid substituents, conversion to carbamates, hydroxylation of aromatic rings, and heteroatom-substitution in aromatic rings.

In another aspect, the present invention features a pharmaceutical composition comprising a compound of formula (I) (or a combination of two or more compounds of formula (I)) and a pharmaceutically acceptable carrier. Also included in the present invention is a medicament composition including any of the compounds of formula (I), alone or in a combination, together with a suitable excipient.

In a further aspect, the invention features a method of inhibiting the A₂ₐ adenosine receptors (e.g., with an Kᵢ value of less than 10 μM; preferably, less than 1 μM in a cell) including the step of contacting the cell with an effective amount of one or more compounds of formula (I). Also with the scope of the invention is a method of modulating the A₂ₐ adenosine receptor signaling pathways in a cell or in a subject (e.g., a mammal such as human), including the step of contacting the cell with or administering to the subject an effective amount of one or more of a compound of formula (I).

Also within the scope of the present invention is a method of treating a subject or preventing a subject from suffering a condition or a disease wherein the causes or symptoms of the condition or disease are associated with an activation of the A₂ₐ adenosine receptor. The method includes the step of administering to the subject an effective amount of one or more of a compound of formula (I). The conditions or diseases can be, e.g., neurodegenerative diseases such as Parkinson's disease and Parkinson's-like syndromes such as progressive supranuclear palsy and multiple system atrophy, senile dementia such as Alzheimer's disease, depression, AIDS encephalopathy, multiple sclerosis, amyotrophic lateral sclerosis, migraine, attention deficit disorder, narcolepsy, sleep apnea or other disorders that cause excessive daytime sleepiness, Huntington's disease, cerebral ischemia, brain trauma, hepatic fibrosis, cirrhosis, and fatty liver.

Compounds of formula (I) may be utilized as sedatives, muscle relaxants, antipsychotics, antidepressants, anxiolytics, analgesics, respiratory stimulants, antiepileptics, anticonvulsants, and cardioprotective agents.

Also within the scope of the invention is a method of treating or preventing a condition or a disease characterized by or resulted from an over-activation of the A₂ₐ adenosine receptor by administering to a subject in need of such a treatment an effective amount of any of compounds of formula (I) in combination with one or more known A₂ₐ antagonists. For example, a patient suffering from Parkinson's disease can be treated by administering an effective amount of a compound of formula (I) in combination with an agent such as L-DOPA, a dopaminergic agonist, an inhibitor of monoamine oxidase (type B), a DOPA decarboxylase inhibitor, or a catechol-O-methyltransferase inhibitor. The compound of formula (I) and the agent can be administered to a patient simultaneously or in sequence. The invention also includes a pharmaceutical composition containing one or more of a compound of formula (I), one or more of a known A₂ₐ antagoinst, and a suitable excipient.

As used herein, an “alkyl” group refers to a saturated aliphatic hydrocarbon group containing 1-8 (e.g., 1-6 or 1-4) carbon atoms. An alkyl group can be straight or branched. Examples of an alkyl group include, but are not limited to, methyl, ethyl, propyl, isopropyl, butyl, isobutyl, sec-butyl, tert-butyl, n-pentyl, n-heptyl, and 2-ethylhexyl. A alkyl group can be optionally substituted with one or more substituents such as alkoxy, cycloalkyloxy, heterocycloalkyloxy, aryloxy, heteroaryloxy, aralkyloxy, heteroarylakloxy, amino, nitro, carboxy, cyano, halo, hydroxy, sulfo, mercapto, alkylsulfanyl, alkylsulfinyl, alkylsulfonyl, aminocarbonyl, alkylcarbonylamino, cycloalkylcarbonylamino, cycloalkyl-alkylcarbonylamino, arylcarbonylamino, aralkylcarbonylamino, heterocycloalkyl-carbonylamino, heterocycloalkyl-alkylcarbonylamino, heteroarylcarbonylamino, heteroaralkylcarbonylamino, urea, thiourea, sulfamoyl, sulfamide, alkoxycarbonyl, or alkylcarbonyloxy. An “alkylene” is a divalent alkyl group, as defined herein.

As used herein, an “alkenyl” group refers to an aliphatic carbon group that contains 2-8 (e.g., 2-6 or 2-4) carbon atoms and at least one double bond. Like an alkyl group, an alkenyl group can be straight or branched. Examples of an alkenyl group include, but are not limited to, allyl, isoprenyl, 2-butenyl, and 2-hexenyl. An alkenyl group can be optionally substituted with one or more substituents such as alkoxy, cycloalkyloxy, heterocycloalkyloxy, aryloxy, heteroaryloxy, aralkyloxy, heteroarylalkoxy, amino, nitro, carboxy, cyano, halo, hydroxy, sulfo, mercapto, alkylsulfanyl, alkylsulfinyl, alkylsulfonyl, aminocarbonyl, alkylcarbonylamino, cycloalkylcarbonylamino, cycloalkyl-alkylcarbonylamino, arylcarbonylamino, aralkylcarbonylamino, heterocycloalkyl-carbonylamino, heterocycloalkyl-alkylcarbonylamino, heteroarylcarbonylamino, heteroaralkylcarbonylamino, urea, thiourea, sulfamoyl, sulfamide, alkoxycarbonyl, or alkylcarbonyloxy. An “alkenylene” is a divalent alkenyl group, as defined herein.

As used herein, an “alkynyl” group refers to an aliphatic carbon group that contains 2-8 (e.g., 2-6 or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as alkoxy, cycloalkyloxy, heterocycloalkyloxy, aryloxy, heteroaryloxy, aralkyloxy, heteroarylalkoxy, amino, nitro, carboxy, cyano, halo, hydroxy, sulfo, mercapto, alkylsulfanyl, alkylsulfinyl, alkylsulfonyl, aminocarbonyl, alkylcarbonylamino, cycloalkylcarbonylamino, cycloalkyl-alkylcarbonylamino, arylcarbonylamino, aralkylcarbonylamino, heterocycloalkyl-carbonylamino, heterocycloalkyl-alkylcarbonylamino, heteroarylcarbonylamino, heteroaralkylcarbonylamino, urea, thiourea, sulfamoyl, sulfamide, alkoxycarbonyl, or alkylcarbonyloxy. An “alkynylene” is a divalent alkynyl group, as defined herein.

As used herein, an “amino” group refers to —NR^{X}R^{Y} wherein each of R^{X} and R^{Y} is independently hydrogen, alkyl, cycloalkyl, (cycloalkyl)alkyl, aryl, aralkyl, heterocycloalkyl, (heterocycloalkyl)alkyl, heteroaryl, or heteroaralkyl. When the term “amino” is not the terminal group (e.g., alkylcarbonylamino), it is represented by —NR^{X}—R^{X} has the same meaning as defined above.

As used herein, an “aryl” group refers to phenyl, naphthyl, or a benzofused group having 2 to 3 rings. For example, a benzofused group includes phenyl fused with one or two C₄₋₈ carbocyclic moieties, e.g., 1, 2, 3, 4-tetrahydronaphthyl, indanyl, or fluorenyl. An aryl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy, alkoxycarbonyl, alkylcarbonyloxy, aminocarbonyl, alkylcarbonylamino, cycloalkylcarbonylamino, (cycloalkyl)alkylcarbonylamino, arylcarbonylamino, aralkylcarbonylamino, (heterocycloalkyl)carbonylamino, (heterocycloalkyl)alkylcarbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, an “aralkyl” group refers to an alkyl group (e.g., a C₁₋₄ alkyl group that is substituted with an aryl group. Both “alkl” and “aryl” have been defined above. An example of an aralkyl group is benzyl.

As used herein, a “cycloalkyl” group refers to an aliphatic carbocyclic ring of 3-10 (e.g., 4-8) carbon atoms. Examples of cycloalkyl groups include cyclopropyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, and bicyclo[3.2.3]nonyl. A “cycloalkenyl” group, as used herein, refers to a non-aromatic carbocyclic ring of 3-10 (e.g., 4-8) carbon atoms having one or more double bond. Examples of cycloalkenyl groups include cyclopentenyl, 1,4-cyclohexa-di-enyl, cycloheptenyl, cyclooctenyl, hexahydro-indenyl, octahydro-naphthyl, bicyclo[2.2.2]octenyl, and bicyclo[3.3.1]nonenyl. A cycloalkyl or cycloalkenyl group can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heterolaroyl, amino, nitro, carboxy, alkoxycarbonyl, alkylcarbonyloxy, aminocarbonyl, alkylcarbonylamino, cycloalkylcarbonylamino, (cycloalkyl)alkylcarbonylamino, arylcarbonylamino, aralkylcarbonylamino, (heterocycloalkyl)carbonylamino, (heterocycloalkyl)alkylcarbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, a “heterocycloalkyl” group refers to a 3- to 10-membered (e.g., 4 to 8-membered) saturated ring structure, in which one or more of the ring atoms is a heteroatom, e.g., N, O, or S. Examples of a heterocycloalkyl group include piperidinyl, piperazinyl, tetrahydropyranyl, tetrahydrofuryl, dioxolanyl, oxazolidinyl, isooxazolidinyl, morpholinyl, octahydro-benzofuryl, octahydro-chromenyl, octahydro-thiochromenyl, octahydro-indolyl, octahydro-pyrindinyl, decahydro-quinolinyl, octahydro-benzo[b]thiophenyl, 2-oxa-bicyclo[2.2.2]octyl, 1-aza-bicyclo[2.2.2]octyl, 3-aza-bicyclo[3.2.1]octyl, anad 2,6-dioxa-tricyclo[3.3.1.0^{3,7}]nonyl. A “heterocycloalkenyl” group, as used herein, refers to a 3- to 10-membered (e.g., 4- to 8-membered) non-aromatic ring structure having one or more double bonds, and wherein one or more of the ring atoms is a heteroatom, e.g., N, O, or S. A heterocycloalkyl or heterocycloalkenyl group can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy, alkoxycarbonyl, alkylcarbonyloxy, aminocarbonyl, alkylcarbonylamino, cycloalkylcarbonylamino, (cycloalkyl)alkylcarbonylamino, arylcarbonylamino, aralkylcarbonylamino, (heterocycloalkyl)carbonylamino, (heterocycloalkyl)alkylcarbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

A “heteroaryl” group, as used herein, refers to a monocyclic, bicyclic, or tricyclic ring structure having 5 to 15 ring atoms wherein one or more of the ring atoms is a heteroatom, e.g., N, O, or S and wherein one ore more rings of the bicyclic or tricyclic ring structure is aromatic. Some examples of heteroaryl are pyridyl, furyl, pyrrolyl, thienyl, thiazolyl, oxazolyl, imidazolyl, indolyl, tetrazolyl, benzofuryl, benzthiazolyl, xanthene, thioxanthene, phenothiazine, dihydroindole, and benzo[1,3]dioxole. A heteroaryl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy, alkoxycarbonyl, alkylcarbonyloxy, aminocarbonyl, alkylcarbonylamino, cycloalkylcarbonylamino, (cycloalkyl)alkylcarbonylamino, arylcarbonylamino, aralkylcarbonylamino, (heterocycloalkyl)carbonylamino, (heterocycloalkyl)alkylcarbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl. A “heteroaralkyl” group, as used herein, refers to an alkyl group (e.g., a C₁₋₄ alkyl group) that is substituted with a heteroaryl group. Both “alkyl” and “heteroaryl” have been defined above.

As used herein, “cyclic moiety” includes cycloalkyl, heterocycloalkyl, cycloalkenyl, heterocycloalkenyl, aryl, or heteroaryl, each of which has been defined previously.

As used herein, an “acyl” group refers to a formyl group or alkyl-C(═O)— where “alkyl” has been defined previously. Acetyl and pivaloyl are examples of acyl groups.

As used herein, a “carbamoyl” group refers to a group having the structure —O—CO—NR^{X}R^{Y} or —NR^{X}—CO—O—R^{Z} wherein R^{X} and R^{Y} have been defined above and R^{Z} is alkyl, cycloalkyl, (cycloalkyl)alkyl, aryl, aralkyl, heterocycloalkyl, (heterocycloalkyl)alkyl, heteroaryl, or heteroaralkyl.

As used herein, a “carboxy” and a “sulfo” group refer to —COOH and —SO₃H, respectively.

As used herein, an “alkoxy” group refers to an alkyl-O— group where “alkyl” has been defined previously.

As used here in, a “sulfoxy” group refers to —O—SO—R^{X} or —SO—R^{X}, where R^{X} has been defined above.

As used herein, a “halogen” or “halo” group refers to fluorine, chlorine, bromine or iodine.

As used herein, a “sulfamoyl” group refers to the structure —SO₂—NR^{X}R^{Y} or —NR^{X}—SO₂—R^{Z} wherein R^{X}, R^{Y}, and R^{Z} have been defined above.

As used herein, a “sulfamide” group refers to the structure —NR^{X}—S(O)₂—NR^{Y}R^{Z} wherein R^{X}, R^{Y}, and R^{Z} have been defined above.

As used herein, a “urea” group refers to the structure —N^{X}—CO—NR^{Y}R^{Z} and a “thiourea” group refers to the structure —NR^{X}—CS—NR^{Y}R^{Z}. R^{X}, R^{Y}, and R^{Z} have been defined above.

As used herein, an effective amount is defined as the amount which is required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, N.Y., 537 (1970). As used herein, “patient” refers to a mammal, including a human.

An antagonist is a molecule that binds to the receptor without activating the receptor. It competes with the endogenous ligand(s) or substrate(s) for binding site(s) on the receptor and, thus inhibits the ability of the receptor to transduce an intracellular signal in response to endogenous ligand binding.

As compounds of formula (I) are antagonists of the A₂ₐ subtype of the adenosine receptors, these compounds are useful in inhibiting the consequences of signal transduction through the adenosine A₂ₐ receptor. Thus, compounds of formula (I) possess the therapuetical utility of treating and/or preventing disorders or diseases for which inhibition of the adenosine A₂ₐ receptor signaling pathways is desirable (e.g., Parkinson's Disease or depression).

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

Other features and advantages of the invention will be apparent from the following detailed description, and from the claims.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable materials and methods are described below. All publications, patent applications, patents, and other references mentioned herein are incorporated by reference in their entirety. In addition, the materials, methods, and examples are illustrative only and are not intended to be limiting.

Synthesis of Compounds of formula (I)

Compounds of formula (I) may be prepared by a number of known methods from commercially available or known starting materials.

In one method, compounds of formula (I) wherein X¹ is a bond can be prepared according to Scheme 1 below. Specifically, the method utilizes as 7-halo-[1,2,4]triazolo[1,5-c]pyrimidin-5-ylamine (e.g., 7-chloro-2-furan-2-yl-[1,2,4]triazolo[1,5-c]pyrimidin-5-ylamine) as the key starting material (II). For reference, see, e.g., U.S. Pat. No. 6,222,035. The starting material (II) wherein B is carbon (e.g., 5-halo-pyrazolo[1,5-c]pyrimidin-7-ylamine) can be prepared according to the method described in Kranz, E. et al., Chemische Berichte 105: 388-405 (1972) and Marei, M. G., Bulletin of the Chemical Society of Japan 66: 1172-1175 (1993). Compound (II) can react with a nucleophile L (L has been defined above; an example of L is 2-aminomethyl-pyrrolidine) to form an intermediate compound (III). The reaction can be conducted in an appropriate solvent such as acetonitrile (CH₃CN), dimethyl sulfoxide (DMSO), or N,N-dimethylformamide (DMF) at a temperature ranging from about 80° C. to 120° C. This intermediate (III) can further react with a compound R¹—Y—X²-LG (where each of R¹, Y and X² has been defined above and LG represents an appropriate leaving group such as halide, mesylate, or tosylate) to form a desired compound of formula (I). See route (A) of Scheme 1 and Examples 1 and 2 below. Alternatively, the intermediate compound (III) can react with an appropriate aldehyde or carboxylic acid to form an abide, which can then undergo reductive amination to form a desired compound of formula (I). Some examples of a reducing agent are sodium triacetoxyborohydride, sodium cyanoborohydride, and borane in THF. See route (B) of Scheme 1 and Examples 3-5 below.

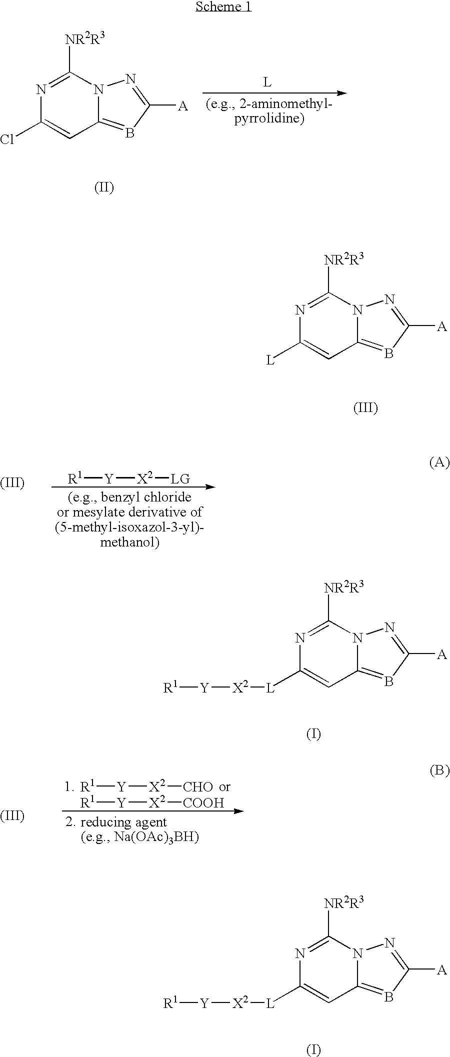

As apparent to a skilled person in the art, protecting groups (e.g., amino protecting group such as Cbz, Fmoc, or Boc) may be needed to avoid undesired side reactions. For reference on protecting groups, see, e.g., Greene and Wutts Protecting Groups in Organic Synthesis, 3^{rd} edition, John Wiley & Sons (1999).

In another method, one can first convert the halo substituent of the starting material compound (II) into an aldehyde substituent. For example, compound (II) can react with aminoacetaldehyde dimethyl acetal to form an intermediate (IV) according to Scheme 2 below. This intermediate (IV) can be treated with trifluoroacetic acid to form the corresponding aldehyde, which can then react with a compound R¹—Y—X²-L′ (where each of R¹, Y, and X² has been defined above and L′ is a precursor of L) to form a compound (I) after undergoing reductive amination using a reagent such as sodium triacetoxyborohydride. See Scheme 2 and Examples 8 and 9.

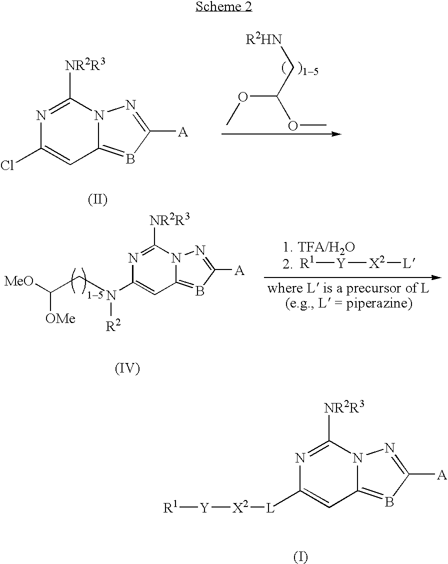

In still another method, a compound of formula (I) wherein X¹ is not a bond can be prepared by reacting starting material compound (II) with an appropriate alkynyl (e.g. a compound of the formula R¹—Y—X²-L-X¹′ (where each of R¹, Y, X², and L has been defined above and X¹′, a precursor of X¹, is an alkynyl; an example of such a compound is 1-(2,4-difluoro-phenyl)-4-prop-2-ynyl-piperazine) to yield a desired compound of formula (I). See Scheme 3 and Examples 6 and 7 below. Such a compound of formula (I) wherein X¹ is an alkynylene can be further modified to form other compounds of formula (I) wherein X¹ is an alkylene by employing an appropriate reducing agent such as 10% Pd on carbon. For reduction of a compound of formula (I) wherein X¹ is an alkynylene to a compound of formula (I) wherein X¹ is an alkenylene, this reaction can be carried out using hydrogenation over Lindlar catalyst, which is 5% Pd on calcium carbonate that has been poisoned with lead (commercially available from Aldrich). Another effective catalyst for the selective hydrogenation would be P2-Ni which could be prepared from nickel acetate and sodium borohydride according to procedure outlined in Hudlicky, M., Reductions in Organic Chemistry, 2nd edition, ACS monograph (1996).

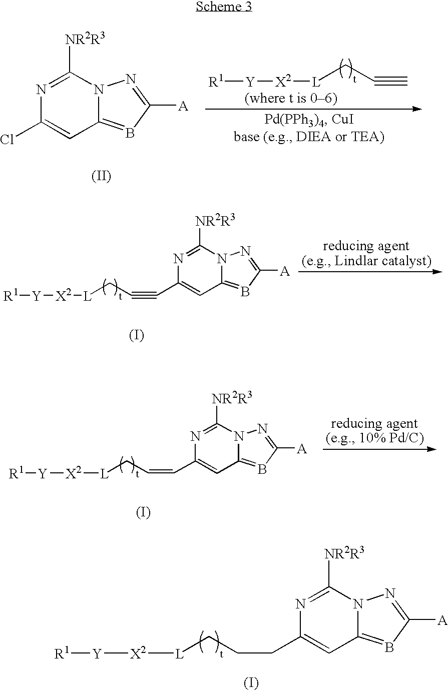

As can be appreciated by the skilled artisan, the above synthetic schemes are exemplary and not intended to comprise a comprehensive list of all means by which the compounds described and claimed in this application may be synthesized. For example, the reaction steps shown in the schemes above can be conducted in a different order, e.g., by reacting a compound of the formula Y-X²-LG with the intermediate compound (III) before coupling with R¹. Further methods will be evident to those of ordinary skill in the art.

Uses for the A₂ₐ Adenosine Antagonist Compounds

Compounds of the invention are useful in the prevention and/or treatment of various neurological diseases and disorders whose causes or symptoms are associated with the A₂ₐ adenosine receptor signaling pathways. Such diseases and disorders include neurodegenerative diseases such as Parkinson's disease and Parkinson's-like syndromes such as progressive supranuclear palsy and multiple system atrophy, Huntington's disease, depression, anxiety, and cerebrovascular disorders such as migraine. In addition, compositions of the invention are useful for neuroprotection, i.e., to prevent or inhibit neuronal death or degeneration associated with conditions such as senile dementia (e.g., Alzheimer's disease), stroke (cerebral ischemia), and brain trauma.

Administration of Compounds of the Invention

Compounds of the invention can be administered to an animal, preferably a mammal, e.g., a human, non-human primate, dog, pig, sheep, goat, cat, mouse, rat, guinea pig, rabbit, hamster, or marmoset. The compounds can be administered in any manner suitable for the administration of pharmaceutical compounds, including, but not limited to, pills, tablets, capsules, aerosols, suppositories, liquid formulations for ingestion or injection or for use as eye or ear drops, dietary supplements, and topical preparations. The compounds can be administered orally, intranasally, transdermally, intradermally, vaginally, intraaurally, intraocularly, buccally, rectally, transmucosally, or via inhalation, implantation (e.g., surgically), or intravenous administration.

Pharmaceutical Compositions

Compounds of the invention can be formulated into pharmaceutical compositions for administration to animals, including humans. These pharmaceutical compositions preferably include a pharmaceutically acceptable carrier and an amount of A₂ₐ adenosine receptor antagonist effective to improve neurological functions such as motor functions and cognitive functions.

Pharmaceutically acceptable carriers useful in these pharmaceutical compositions include, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention can be administered parenterally, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention can be aqueous or oleaginous suspension. These suspensions may be formulated according to techniques known in the art using suitable dispersing or wetting agents and suspending agents. The sterile injectable preparation can also be a sterile injectable solution or suspension in a non-toxic parenterally-acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions also can contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms also can be used for the purposes of formulation.

Parenteral formulations can be a single bolus dose, an infusion or a loading bolus dose followed with a maintenance dose. These compositions can be administered once a day or on an “as needed” basis.

The pharmaceutical compositions of this invention be administered orally in any orally acceptable dosage form including, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents can also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically. Topical application can be effected in a rectal suppository formulation (see above) or in a suitable enema formulation. Topically-transdermal patches may also be used.

For topical applications, the pharmaceutical compositions can be formulated in a suitable ointment containing the active component suspended or dissolved in one or more carriers. Carriers for topical administration of the compounds of this invention include, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention also can be administered by nasal aerosol or inhalation. Such compositions can be prepared according to techniques known in the art of pharmaceutical formulation, and can be prepared as solutions in saline, employing benzyl alcohol or other suitable preservatives, absorption promoters to enhance bioavailability, fluorocarbons, and/or other conventional solubilizing or dispersing agents.

The amount of A₂ₐ adenosine receptor antagonist that may be combined with the carrier materials to produce a single dosage form will vary depending upon the host treated and the particular mode of administration. The compositions can be formulated so that a dosage of between 0.01-100 mg/kg body weight of the A₂ₐ adenosine receptor antagonist is administered to a patient receiving these compositions. In some embodiments of the invention, the dosage is 0.1-10 mg/kg body weight. The composition may be administered as a single dose, multiple doses or over an established period of time in an infusion.

A specific dosage and treatment regimen for any particular patient will depend upon a variety of factors, including the particular A₂ₐ adenosine receptor antagonist, the patient's age, body weight, general health, sex, and diet, and the time of administration, rate of excretion, drug combination, and the severity of the particular disease being treated. Judgment of such factors by medical caregivers is within ordinary skill in the art. The amount of antagonist will also depend on the individual patient to be treated, the route of administration, the type of formulation, the characteristics of the compound used, the severity of the disease, and the desired effect. The amounts of antagonist can be determined by pharmacological and pharmacokinetic principles well-known in the art.

The invention will be further described in the following examples, which do not limit the scope of the invention described in the claims.

In the following examples, unless indicated otherwise, all commercial reagents were obtained from Sigma-Aldrich (St. Louis, Mo.), Lancaster (Windham, N.H.), Acros (Pittsburgh, Pa.), Alfa (Berkshire, UK), TCI (Portland, Oreg.), or Maybridge (Cornwall, UK).

### EXAMPLE 1

### 2-Furan-2-yl-N⁷-[1-(5-methyl-isoxazol-3-ylmethyl)-pyrrolidin-2-ylmethyl]-[1,2,4]triazolo[1,5-c]pyrimidine-5,7-diamine

Synthesis of the title compound is described in subparts (a)-(c) below.

### (a) Methanesulfonic acid 5-methyl-isoxazol-3-ylmethyl ester

(5-Methyl-isoxazol-3-yl)-methanol (57 mg, 0.5 mmol) was dissolved in 4 mL of CH₂Cl₂ along with 1.3 eq. of Et₃N. The solution was cooled in an ice bath and methanesulfonyl chloride (1.2 eq) was added. The reaction mixture was warmed to room temperature and stirred for 45 minutes. It was then quenched with brine and the two layers were separated. The organic layer was dried with Na₂SO₄ and concentrated under reduced pressure to afford the title mesylate derivative.

### (b) 2-Furan-2-yl-N⁷-pyrrolidin-2-ylmethyl-[1,2,4]triazolo[1,5-c]pyrimidine-5,7-diamine

500 mg (2.12 mmol) of 7-chloro-2-furan-2-yl-[1,2,4]triazolo[1,5-c]pyrimidin-5-ylamine (prepared as described in U.S. Pat. No. 6,222,035) was dissolved in 12 mL of DMSO along with 480 mg (3.18 mmol) of CsF and 850 mg (4.24 mmol) of (R)-2-aminomethyl-1-Boc-pyrrolidine (Astatech, Monmouth Junction, N.J.). The reaction mixture was stirred at 110° C. for 18 hours. It was then cooled to room temperature and diluted with CH₂Cl₂. The organic layer was washed with H₂O, dilute 1 M citric acid, brine, dried with Na₂SO₄ and concentrated under reduced pressure. Purification by chromatography (2:1 EtOAc/hexanes) afforded 480 mg of the BOC-protected amine. This material was dissolved in 10 mL of 25% TFA in CH₂Cl₂ and was allowed to stand at room temperature for 4 hours. It was then concentrated under reduced pressure to afford the TFA salt of 2-furan-2-yl-N⁷-pyrrolidin-2-ylmethyl-[1,2,4]triazolo[1,5-c]pyrimidine-5,7-diamine. MS: m/z 300 [M+H]⁺.

### (c) 2-Furan-2-yl-N⁷-[1-(5-methyl-isoxazol-3-ylmethyl)-pyrrolidin-2-ylmethyl]-[1,2,4]triazo[1,5-c]pyrimidine-5,7-diamine

The TFA salt of furan-2-yl-N⁷-pyrrolidin-2-ylmethyl-[1,2,4]triazolo[1,5-c]pyrimidine-5,7-diamine (0.4 mmol; see subpart (b) above) was dissolved in 3 mL of CH₃CN along with Et₃N (2 eq) and methanesulfonic acid 5-methyl-isoxazol-3-ylmethyl ester (1.5 eq; see subpart (a) above). The reaction mixture was stirred at room temperature for 18 hours. It was then concentrated and the resulting crude product was purified by preparative HPLC using a mixture of aqueous, CH₃CN buffered with 0.1% TFA. ¹H NMR (DMSO-d₆) δ 7.60 (d, J=1.0 Hz, 1H), 7.28 (br s, 2H), 7.22 (d, J=3.6 Hz, 1H), 6.68 (dd, J=3.6 Hz, 1.0 Hz, 1H), 6.3 (s, 1H), 5.2 (s, 1H), 3.8 (br s, 2H), 2.3-3.4 (m, 8H), 1.5 (br s, 3H). MS: m/z: 395 [M+H]⁺.

### EXAMPLE 2

### 2-Furan-2-yl-N⁷-methyl-N⁷-[1-(5-methyl-isoxazol-3-ylmethyl)-pyrrolidin-2-ylmethyl]-[1,2,4]triazolo[1,5-c]pyrimidine-5,7-diamine

Synthesis of the title compound is described in subparts (a)-(c) below.

### (a) (R)-2-methylaminomethyl-1-Boc-pyrrolidine

(R)-Boc-proline (4.8 g, 22.3 mmol) was suspended in 100 mL of THF. 1-Ethyl-3-(3-dimethylaminopropyl)carbodiimide (5.13 g, 1.2 eq) was then added to the solution, followed by 1-hydroxybenzotriazole (3.62 g, 1.2 eq) and N-methylmorpholine (3.7 mL, 1.5 eq). The reaction mixture was stirred at room temperature for 30 minutes and 35 mL of methylamine in THF (2.0 M, 3 eq) was added. The reaction mixture was stirred at room temperature for 18 hours. It was then concentrated and the residue was taken up in CH₂Cl₂ and washed with diluted NaHCO₃, water, citric acid (1 N), and brine, dried with Na₂SO₄ and concentrated to yield 4.8 g of the crude carboxamide intermediate. This material was dissolved in 100 mL of anhydrous THF and cooled to 0° C. Borane.THF (53 mL of the 1.0 M solution, 2.5 eq) was added and the reaction mixture was allowed to warm to room temperature and stirred at room temperature for 18 hours. It was then cooled to 0° C. and carefully quenched with 50 mL of methanol. The reaction mixture was concentrated under reduced pressure. The resulting residue was redissolved in 50 mL of methanol and 100 mL of ethyl acetate and concentrated under reduced pressure. The trituration and concentration under reduced pressure were repeated three more times to afford essentially quantitative yield of (R)-2-methylaminomethyl-1-Boc-pyrrolidine, which was then used in the next step without further purification.

### (b) 2-Furan-2-yl-N⁷-methyl-N⁷-pyrrolidin-2-ylmethyl-[1,2,4]triazolo[1,5-c]pyrimidine-5,7-diamine

The intermediate 2-furan-2-yl-N⁷-methyl-N⁷-pyrrolidin-2-ylmethyl-[1,2,4]triazolo[1,5-c]pyrimidine-5,7-diamine was prepared in the same manner as described in Example 1(b) above, except that (R)-2-methylaminomethyl-1-Boc-pyrrolidine (see subpart (a) above) was used as the staring material instead of the commercial reagent (R)-2-aminomethyl-1-Boc-pyrrolidine.

### (c) 2-Furan-2-yl-N⁷-methyl-N⁷-[1-(5-methyl-isoxazol-3-ylmethyl)-pyrrolidin-2-ylmethyl]-[1,2,4]triazolo[1,5-c]pyrimidine-5,7-diamine

Using the same procedure as described in Example 1(c) above, 2-furan-2-yl-N⁷-methyl-N⁷-pyrrolidin-2-ylmethyl-[1,2,4]triazolo[1,5-c]pyrimidine-5,7-diamine (see subpart (b) above) was subjected to the same alkylation reaction. The title product, 2-furan-2-yl-N⁷-methyl-N⁷-[1-(5-methyl-isoxazol-3-ylmethyl)-pyrrolidin-2-ylmethyl]-[1,2,4]triazolo[1,5-c]pyrimidine-5,7-diamine, was obtained after purification by preparative HPLC using a mixture of aqueous CH₃CN buffered with 0.1% TFA. ¹H NMR (DMSO-d₆) δ 7.60 (d, J=1.0 Hz, 1H), 7.28 (br s, 2H), 7.22 (d, J=3.6 Hz, 1H), 6.68 (dd, J=3.6 Hz, 1.0 Hz, 1H), 6.3 (s, 1H), 5.4 (s, 1H), 3.8 (br s, 2H), 2.5 (s, 3H), 2.2-3.2 (m, 8H), 1.6 (br s, 3H). MS: m/z: 409 [M+H]⁺.

### EXAMPLE 3

### N⁷-[1-(2-Chloro-6-fluoro-benzyl)-pyrrolidin-2-ylmethyl]-2-furan-2-yl-[1,2,4]triazolo[1,5-c]pyrimidine-5,7-diamine

The TFA salt of 2-furan-2-yl-N⁷-pyrrolidin-2-ylmethyl-[1,2,4]triazolo[1,5-c]pyrimidine-5,7-diamine (0.3 mmol; see Example 1(b) above) was dissolved in 5 mL of CH₂Cl₂ along with 1 eq of Et₃N. 2-Chloro-6-fluorobenzaldehyde (1.2 eq) was then added to the solution, followed by sodium triacetoxyborohydride (2.5 eq). The reaction mixture was stirred at room temperature for 18 hours. It was then concentrated and the resulting crude product was purified by preparative HPLC using a mixture of aqueous CH₃CN buffered with 0.1% TFA. ¹H NMR (DMSO-d₆) δ 7.60 (d, J=1.0 Hz, 1H), 7.28 (br s, 2H), 7.22 (d, J=3.6 Hz, 1H), 7.0-7.3 (m, 3H), 6.68 (dd, J=3.6 Hz, 1.0 Hz, 1H), 5.4 (s, 1H), 3.8 (br s, 2H), 2.2-3.2 (m, 8H). MS: m/z: 443 [M+H]⁺.

### EXAMPLE 4

### N⁷-[1-(2,6-Difluoro-benzyl)-piperidin-2-ylmethyl]-2-furan-2-yl-N⁷-methyl-[1,2,4]triazolo[1,5c]pyrimidine-5,7-diamine

The title compound was prepared according to the same procedure as described in Example 2 above, except that (R)-2-methylaminomethyl-1-Boc-pyrrolidine was replaced with (R)-2-methylaminomethyl-1-Boc-piperidine, which was prepared according to the procedure outlined in Example 2(a) using N-Boc-piperidine-2-carboxylic acid as the starting material. N⁷-[1-(2,6-Difluoro-benzyl)-piperidin-2-ylmethyl]-2-furan-2-yl-N⁷-methyl-[1,2,4]triazolo[1,5-c]pyrimidine-5,7-diamine was obtained after purification by preparative HPLC using a mixture of aqueous CH₃CN buffered with 0.1% TFA. ¹H NMR (DMSO-d₆) δ 7.60 (d, J=1.0 Hz, 1H), 7.28 (br s, 2H), 7.22 (d, J=3.6 Hz, 1H), 7.0-7.3 (m, 3H), 6.68 (dd, J=3.6 Hz, 1.0 Hz, 1H), 5.4 (s, 1H), 3.8 (br s, 2H), 2.2-3.2 (m, 10H). MS: m/z: 454 [M+H]⁺.

### EXAMPLE 5

### N⁷-[1-(2-Fluoro-benzyl)-pyrrolidin-2-ylmethyl]-2-furan-2-yl-N⁷-methyl-[1,2,4]triazolo[1,5-c]pyrimidine-5,7-diamine

The title compound was prepared in the same manner as described in Example 3 above, except that 2-furan-2-yl-N⁷-pyrrolidin-2-ylmethyl-[1,2,4]triazolo[1,5-c]pyrimidine-5,7-diamine was replaced with 2-furan-2-yl-N⁷-methyl-N⁷-pyrrolidin-2-ylmethyl-[1,2,4]triazolo[1,5-c]pyrimidine-5,7-diamine (prepared according to the procedure as described in Example 2(b) above) and 2-chloro-6-fluorobenzaldehyde was replaced with 2-fluorobenzaldehyde. N⁷-[1-(2-fluoro-benzyl)-pyrrolidin-2-ylmethyl]-2-furan-2-yl-N⁷-methyl-[1,2,4]triazolo[1,5-c]pyrimidine-5,7-diamine was obtained after purification by preparative HPLC using a mixture of aqueous CH₃CN buffered with 0.1% TFA. ¹H NMR (DMSO-d₆) δ 7.60 (d, J=1.0 Hz, 1H), 7.28 (br s, 2H), 7.22 (d, J=3.6 Hz, 1H), 6.8-7.3 (m, 4H), 6.68 (dd, J=3.6 Hz, 1.0 Hz, 1H), 5.4 (s, 1H), 3.8 (br s, 2H), 2.2-3.2 (m, 8H). MS: m/z: 422 [M+H]⁺.

### EXAMPLE 6

### 7-{3-[4-(2,4-Difluoro-phenyl)-piperazin-1-yl]-prop-1-ynyl}-2-furan-2-yl-[1,2,4]triazolo[1,5-c]pyrimidin-5-ylamine

Synthesis of the title compound is described in subparts (a) and (b) below.

### (a) 1-(2,4-Difluoro-phenyl)-4-prop-2-ynyl-piperazine

1-(2,4-Difluoro-phenyl)-piperazine (3.3 mmol; prepared by reacting piperazine with 1-bromo-2,4-difluorobenzene according to the procedure described in WO 01/92264) was dissolved in 20 mL of THF and 1.1 eq. of propargyl bromide was added, followed by [how much] eq. of anhydrous K₂CO₃. The reaction mixture was stirred at room temperature for 18 hours. It was then diluted with EtOAc and washed with brine, dried with Na₂SO₄ and concentrated to afford 1-(2,4-difluoro-phenyl)-4-prop-2-ynyl-piperazine.

### (b) 7-{3-[4-(2,4-Difluoro-phenyl)-piperazin-1-yl]-prop-1-ynyl}-2-furan-2-yl-[1,2,4]triazolo[1,5-c]pyrimidin-5-ylamine

1-(2,4-Difluoro-phenyl)-4-prop-2-ynyl-piperazine (300 mg, 1.27 mmol, 1.5 eq; see subpart (a) above) was added to 200 mg of 7-chloro-2-furan-2-yl-[1,2,4]triazolo[1,5-c]pyrimidin-5-ylamine (0.85 mmol, 1 eq; see Example 1(b) above) along with 6 mL of anhydrous DMF in a sealed reaction tube. After addition of Pd(PPh₃)₄ (150 mg, 15 mol %), CuI (26 mg, 15 mol %), PPh₃ (33 mg, 15 mol %) and Et₃N (0.6 mL, 5 eq), the reaction mixture was purged with N₂, sealed, and stirred at 110° C. for 18 hours. It was then cooled to room temperature and purified by preparative HPLC to afford 7-{3-[4-(2,4-difluoro-phenyl)-piperazin-1-yl]-prop-1-ynyl}-2-furan-2-yl-[1,2,4]triazolo[1,5-c]pyrimidin-5-ylamine. ¹H NMR (DMSO-d₆) δ 7.60 (d, J=1.0 Hz, 1H), 7.28 (br s, 2H), 7.22 (d, J=3.6 Hz, 1H), 6.8-7.3 (m, 3H), 6.68 (dd, J=3.6 Hz, 1.0 Hz, 1H), 6.5 (s, 1H), 3.1 (br s, 2H), 2.4-3.6 (m, 8H). MS: m/z: 436 [M+H]⁺.

### EXAMPLE 7

### 7-{3-[4-(2,4-Difluoro-phenyl)-piperazin-1-yl]-propyl}-2-furan-2-yl-[1,2,4]triazolo[1,5-c]pyrimidin-5-ylamine

7-{3-[4-(2,4-Difluoro-phenyl)-piperazin-1-yl]-prop-1-ynyl}-2-furan-2-yl-[1,2,4]triazolo[1,5-c]pyrimidin-5-ylamine (50 mg; see Example 6 above) was dissolved in 5 mL 1:1 THF/MeOH. 10% Palladium on carbon (10 mg) was added, and the reaction mixture was hydrogenated under 1 atm of H₂, at room temperature for 30 minutes. The catalyst was filtered and the reaction mixture was concentrated to afford 7-{3-[4-(2,4-difluoro-phenyl)-piperazin-1-yl]-propyl}-2-furan-2-yl-[1,2,4]triazolo[1,5-c]pyrimidin-5-ylamine. ¹H NMR (DMSO-d₆) δ 7.60 (d, J=1.0 Hz, 1H), 7.28 (br s, 2H), 7.22 (d, J=3.6 Hz, 1H), 6.8-7.3 (m, 3H), 6.68 (dd, J=3.6 Hz, 1.0 Hz, 1H), 6.5 (s, 1H), 3.1 (br s, 2H), 2.2-3.6 (m, 14H). MS: m/z: 440 [M+H]⁺.

### EXAMPLE 8

### N⁷-{2-[4-(2,4-Difluoro-phenyl)-piperazin-1-yl]-ethyl}-2-furan-2-yl-[1,2,4]triazolo[1,5-c]pyrimidine-5,7-diamine

7-Chloro-2-furan-2-yl-[1,2,4-triazolo[1,5-c]pyrimidin-5-ylamine (1 g; see Example 1(b) above) was suspended in 20 mL of DMSO along with 1.5 eq of CsF and 5 eq of aminoacetaldyde dimethyl acetal. The reaction mixture was stirred at 110° C. for 18 hours. It was then cooled to room temperature and diluted with EtOAc and washed with H₂O and brine, dried with Na₂SO₄ and concentrated to afford N⁷-(2,2-dimethoxy-ethyl)-2-furan-2-yl-[1,2,4]triazolo[1,5-c]pyrimidine-5,7-diamine. This dimethyl acetal intermediate (40 mg, 0.13 mmol) was then unmasked to the corresponding aldehyde by suspending in a solution of 2 mL of CH₂Cl₂ and 0.2 mL of 2:1 solution of TFA/H₂O. The resulting reaction mixture was stirred at room temperature for 4 hours. It was then neutralized with 0.25 mL of Et₃N. 1-(2,4-Difluoro-phenyl)-piperazine (40 mg, 1.5 eq; see Example 6(a) above) was added, followed by 140 mg of Na(OAc)₃BH. The resulting reaction mixture was stirred at room temperature for 2 hours. It was then concentrated and then purified by preparative HPLC to afford the title compound. ¹H NMR (DMSO-d₆) δ 7.60 (d, J=1.0 Hz, 1H), 7.28 (br s, 2H), 7.22 (d, J=3.6 Hz, 1H), 6.8-7.3 (m, 3H), 6.68 (dd, J=3.6 Hz, 1.0 Hz, 1H), 6.5 (s, 1H), 3.1 (br s, 2H), 2.2-3.6 (m, 12H). MS: m/z: 441 [M+H]⁺.

### EXAMPLE 9

### N⁷-{2-[4-(2,4-Difluoro-phenyl)-piperazin-1-yl]-ethyl}-2-furan-2-yl-N⁷-methyl-[1,2,4]triazolo[1,5-c]pyrimidine-5,7-diamine

N⁷-{2-[4-(2,4-Difluoro-phenyl)-piperazin-1-yl]-ethyl}-2-furan-2-yl-N⁷-methyl-[1,2,4]triazolo[1,5-c]pyrimidine-5,7-diamine was prepared in the same manner as described in Example 8 above, except that N-methylaminoacetaldehyde dimethyl acetal was used instead of aminoacetaldyde dimethyl acetal. The title compound was obtained after purification by preparative HPLC. ¹H NMR(DMSO-d₆) δ 7.60 (d, J=1.0 Hz; 1H), 7.28 (br s, 2H), 7.22 (d, J=3.6 Hz, 1H), 6.8-7.3 (m, 3H), 6.68 (dd, J=3.6 Hz, 1.0 Hz, 1H), 6.5 (s, 1H), 3.1 (br s, 2H), 2.6 (s, 3H), 2.2-3.6 (m, 12H). MS: m/z: 455 [M+H]⁺.

The compounds listed in the following table were prepared in an analogous manner as described in the methods and examples above. The mass spectroscopy data of these compounds are included in the table.

| | | Mass Spec. | Synthetic |
|---|---|---|---|
| Example | Compound Name | (m/z) | Method |
| Ex. 9 | 2-Furan-2-yl-N⁷-(1-pyridin-4-ylmethyl-pyrrolidin-2- | 391 | Ex. 1 |
| | ylmethyl)-[1,2,4]triazolo[1,5-c]pyrimidine-5,7-diamine | [M + H]+ | |
| Ex. 10 | N⁷-[1-(2,6-Dichloro-pyridin-4-ylmethyl)-pyrrolidin-2- | 460 | Ex. 1 |
| | ylmethyl]-2-furan-2-yl-[1,2,4]triazolo[1,5-c]pyrimidine-5,7- | [M + H]+ | |
| | diamine | | |
| Ex. 11 | N⁷-[1-(2-Chloro-pyridin-4-ylmethyl)-pyrrolidin-2-ylmethyl]- | 426 | Ex. 1 |
| | 2-furan-2-yl-[1,2,4]triazolo[1,5-c]pyrimidine-5,7-diamine | [M + H]+ | |
| Ex. 12 | N⁷-[1-(2,3-Difluoro-benzyl)-pyrrolidin-2-ylmethyl]-2-furan- | 426 | Ex. 3 |
| | 2-yl-[1,2,4]triazolo[1,5-c]pyrimidine-5,7-diamine | [M + H]+ | |
| Ex. 13 | 2-Furan-2-yl-N⁷-methyl-N⁷-[1-(2,3,6-trifluoro-benzyl)- | 472 | Ex. 4 |
| | piperidin-2-ylmethyl]-[1,2,4]triazolo[1,5-c]pyrimidine-5,7- | [M + H]+ | |
| | diamine | | |
| Ex. 14 | N⁷-[1-(2,4-Difluoro-benzyl)-piperidin-2-ylmethyl]-2-furan-2- | 454 | Ex. 4 |
| | yl-N7-methyl-[1,2,4]triazolo[1,5-c]pyrimidine-5,7-diamine | [M + H]+ | |
| Ex. 15 | N⁷-[1-(5-Chloro-furan-2-ylmethyl)-pyrrolidin-2-ylmethyl]-2- | 429 | Ex. 5 |
| | furan-2-yl-N7-methyl-[1,2,4]triazolo[1,5-c]pyrimidine-5,7- | [M + H]+ | |
| | diamine | | |
| Ex. 16 | N⁷-(1-Benzofuran-2-ylmethyl-pyrrolidin-2-ylmethyl)-2- | 444 | Ex. 5 |
| | furan-2-yl-N7-methyl-[1,2,4]triazolo[1,5-c]pyrimidine-5,7- | [M + H]+ | |
| | diamine | | |
| Ex. 17 | N⁷-[1-(5-Chloro-1-methyl-3-trifluoromethyl-1H-pyrazol-4- | 511 | Ex. 5 |
| | ylmethyl)-pyrrolidin-2-ylmethyl]-2-furan-2-yl-N7-methyl- | [M + H]+ | |
| | [1,2,4]triazolo[1,5-c]pyrimidine-5,7-diamine | | |
| Ex. 18 | N⁷-[1-(2,3-Difluoro-benzyl)-pyrrolidin-2-ylmethyl]-2-furan- | 440 | Ex. 5 |
| | 2-yl-N7-methyl-[1,2,4]triazolo[1,5-c]pyrimidine-5,7-diamine | [M + H]+ | |
| Ex. 19 | 2-Furan-2-yl-N⁷-methyl-N⁷-(1-pyridin-2-ylmethyl-pyrrolidin- | 405 | Ex. 5 |
| | 2-ylmethyl)-[1,2,4]triazolo[1,5-c]pyrimidine-5,7-diamine | [M + H]+ | |
| Ex. 20 | 2-Furan-2-yl-N⁷-methyl-N⁷-(1-pyridin-3-ylmethyl-pyrrolidin- | 405 | Ex. 5 |
| | 2-ylmethyl)-[1,2,4]triazolo[1,5-c]pyrimidine-5,7-diamine | [M + H]+ | |
| Ex. 21 | 2-Furan-2-yl-N⁷-methyl-N⁷-(1-pyridin-4-ylmethyl-pyrrolidin- | 405 | Ex. 5 |
| | 2-ylmethyl)-[1,2,4]triazolo[1,5-c]pyrimidine-5,7-diamine | [M + H]+ | |
| Ex. 22 | N⁷-[1-(6-Chloro-pyridin-3-ylmethyl)-pyrrolidin-2-ylmethyl]- | 440 | Ex. 2 |
| | 2-furan-2-yl-N7-methyl-[1,2,4]triazolo[1,5-c]pyrimidine-5,7- | [M + H]+ | |
| | diamine | | |
| Ex. 23 | 2-Furan-2-yl-N⁷-methyl-N⁷-[1-(2,3,5,6-tetrafluoro-benzyl)- | 476 | Ex. 5 |
| | pyrrolidin-2-ylmethyl]-[1,2,4]triazolo[1,5-c]pyrimidine-5,7- | [M + H]+ | |
| | diamine | | |
| Ex. 24 | 1-(5-Amino-2-furan-2-yl-[1,2,4]triazolo[1,5-c]pyrimidin-7- | 309 | Ex. 6 |
| | ylethynyl)-cyclopentanol | [M + H]+ | |
| Ex. 25 | 1-(5-Amino-2-furan-2-yl-[1,2,4]triazolo[1,5-c]pyrimidin-7- | 324 | Ex. 6 |
| | ylethynyl)-cyclohexanol | [M + H]+ | |
| Ex. 26 | 4-(5-Amino-2-furan-2-yl-[1,2,4]triazolo[1,5-c]pyrimidin-7- | 346 | Ex. 6 |
| | yl)-2-phenyl-but-3-yn-2-ol | [M + H]+ | |
| Ex. 27 | 7-(3-Cyclohexyl-prop-1-ynyl)-2-furan-2-yl- | 322 | Ex. 6 |
| | [1,2,4]triazolo[1,5-c]pyrimidin-5-ylamine | [M + H]+ | |
| Ex. 28 | 2-Furan-2-yl-N⁷-{2-[4-(2,4,6-trifluoro-phenyl)-piperazin-1- | 459 | Ex. 8 |
| | yl]-ethyl}-[1,2,4]triazolo[1,5-c]pyrimidine-5,7-diamine | [M + H]+ | |
| Ex. 29 | N⁷-{2-[4-(2-Fluoro-phenyl)-piperazin-1-yl]-ethyl}-2-furan-2- | 423 | Ex. 8 |
| | yl-[1,2,4]triazolo[1,5-c]pyrimidine-5,7-diamine | [M + H]+ | |
| Ex. 30 | N⁷-{2-[4-(2,5-Difluoro-phenyl)-piperazin-1-yl]-ethyl}-2- | 441 | Ex. 8 |
| | furan-2-yl-[1,2,4]triazolo[1,5-c]pyrimidine-5,7-diamine | [M + H]+ | |
| Ex. 31 | 2-Furan-2-yl-N⁷-methyl-N⁷-[2-(4-phenyl-piperazin-1-yl)- | 419 | Ex. 9 |
| | ethyl]-[1,2,4]triazolo[1,5-c]pyrimidine-5,7-diamine | [M + H]+ | |
| Ex. 32 | 2-Furan-2-yl-N⁷-methyl-N⁷-{2-[4-(2,4,6-trifluoro-phenyl)- | 473 | Ex. 9 |
| | piperazin-1-yl]-ethyl}-[1,2,4]triazolo[1,5-c]pyrimidine-5,7- | [M + H]+ | |
| | diamine | | |

The A₂ₐ modulating activity of compounds of the present invention can be assessed by methods described in the following examples.

### EXAMPLE 33

Numerous compounds of the present invention were prepared (see working examples and table above) and tested. Specifically, the Kᵢ values for rat A₁ adenosine receptors and for rat A₂ₐ adenosine receptors were determined according to the following binding assay protocol. The ratio A₂ₐ/A₁ was also calculated.

Materials

Adenosine deaminase and HEPES Were purchased from Sigma-Aldrich (St. Louis, Mo.). Ham's F-12 cell culture medium and fetal bovine serum were purchased from GIBCO Life Technologies (Gaithersburg, Md.). Antibiotic G418, Falcon 150 mM culture plates and Costar 12-well culture plates were purchased from Fisher (Pittsburgh, Pa.). [³H]CPX was purchased from DuPont-New England Nuclear Research Products (Boston, Mass.). Penicillin/streptomycin antibiotic mixture was purchased from Mediatech (Washington, D.C.). The composition of HEPES-buffered Hank's solution was: 130 mM NaCl, 5.0 mM Cl, 1.5 mM CaCl₂, 0.41 mM MgSO₄, 0.49 mM Na₂HPO₄, 0.44 mM KH₂PO₄, 5.6 mM dextrose, and 5 mM HEPES (pH 7.4).

Membrane Preparation

A₂ₐ Receptor: Membranes were prepared from rat brain tissues purchased from Pel-Freez. Tissues were homogenized in buffer A (10 mM EDTA, 10 mM Na-HEPES, pH 7.4) supplemented with protease inhibitors (10 μg/m benzamidin 100 μM and 2 μg/ml each of aprotinin, pepstatin and leupeptin), and centrifuged at 20,000×g for 20 minutes. Pellets were resuspended and washed twice with buffer HE (10 mM Na-HEPES, 1 mM EDTA, pH 7.4, plus protease inhibitors). Final pellets were resuspended in buffer HE, supplemented with 10% (w/v) sucrose and protease inhibitors, and frozen in aliquots at −80° C. Protein concentrations were measured using BCA protein assay kit (Pierce).

A₁ Receptor: Membranes were prepared from rat cerebral cortex isolated from freshly euthanized rats. Tissues were homogenized in buffer A (10 mM EDTA, 10 mM Na-HEPES, pH 7.4) supplemented with protease inhibitors (10 μg/ml benzamidine, 100 μM PMSF, and 2 μg/ml each of aprotinin, pepstatin and leupeptin), and centrifuged at 20,000×g for 20 minutes. Pellets were resuspended and washed twice with buffer HE (10 mM Na-HEPES, 1 mM EDTA, pH 7.4, plus protease inhibitors). Final pellets were resuspended in buffer HE, supplemented with 10% (w/v) sucrose and protease inhibitors, and frozen in aliquots at −80° C. Protein concentrations were measured using BCA protein assay kit (Pierce).

Radioligand Binding Assays

Membranes (40-70 μg membrane protein), radioligands and varying concentrations of test compounds of the present invention were incubated in triplicates in 0.1 ml buffer HE plus 2 units/ml adenosine deaminase for 2.5 hours at 21° C. Radioligand [³H]DPCPX was used for competition binding assays on A₁ receptors and [³H]ZM241385 was used for A₂ₐ adenosine receptors. Nonspecific binding was measured in the presence of 10 μM NECA for A₁ receptors, or 10 μM XAC for A₂ₐ receptors. Binding assays were terminated by filtration over Whatman GF/C glass fiber filters using a BRANDEL cell harvester. Filters were rinsed three times with 3-4 mL ice cold 10 mM Tris-HCl, pH 7.4 and 5 mM MgCl₂ at 4° C., and were counted in a Wallac β-counter.

Analysis of Binding Data

Kᵢ determination: Competition binding data were fit to a single-site binding model and plotted using Prizm GraphPad. Cheng-Prusoff equation Kᵢ=IC₅₀/(1+[I]/K_{d}) was used to calculate Kᵢ values from IC₅₀ values, where Kᵢ is the affinity constant for the competing test compound, [I] is the concentration of the free radioligand, and K_{d} is the affinity constant for the radioligand.

A₂ₐ % binding: Data were generally expressed as percentage of total specific binding at 1 μM of competing test compound (% total specific binding)=100% ×(specific binding with 1 μM of competing test compound/total specific binding).

Results

Compounds of the present invention typically exhibited Kᵢ values of less than 10 μM and A₂ₐ % binding ranging from 1% to 50%; some compounds exhibited Kᵢ values of less than 1 μM.

### EXAMPLE 34

Catalepsy Experiments

Haloperidol-induced catalepsy was used to mimic the effects of Parkinson's disease in rats and mice. Animals were injected with haloperidol, which causes immobility. A test compound of the present invention was then administered orally and the compound's ability to reverse these Parkinson's-like symptoms was analyzed. For reference, see Sanberg et al., Behavioral Neuroscience 102: 748-759 (1988).

Rats

Male Sprague-Dawley rats (225-275 g) were injected with haloperidol (1 mg/kg s.c.) to induce catalepsy. These rats were then subjected to the bar test. In this test, the rats' forelimbs were placed on an aluminum bar (1 cm in diameter) suspended horizontally 10 cm above the surface of the bench. The elapsed time until the rat placed one forepaw back on the bench was measured, with a maximum time of 120 seconds allowed. It should be noted that these rats were in a cataleptic state and therefore were unable to correct an externally imposed posture (i.e., the cataleptic rats, when placed in this unnatural position, were unable to come down from the horizontal bar over a period of 120 seconds or more). Once the rats showed a stable baseline cataleptic response (about three hours after haloperidol injection), a test compound of the present invention or vehicle alone is administered orally, and catalepsy data from the bar test were measured every 30 minutes for the next 3 hours. Data were analyzed by one factor analysis of variance with Dunnett's ‘t’ test used to make post-hoc comparisons. Many compounds of this invention showed oral activity at a dosage of 10 mg/kg or lower, which allowed the cataleptic animals to come down from the bar within 60 seconds and remained in a catalepsy-free state for at least 60 minutes.

Mice

Mice catalepsy experiment was conducted in the same manner as described above except mice (CD-1; 25-30 g) were used instead of rats, the dose of haloperidol was 3 mg/kg s.c. instead of 1 mg/kg s.c., and the bar was suspended 4.5 cm instead of 10 cm above the surface of the bench. Many compounds of this invention showed oral activity at a dosage of 10 mg/kg or lower, which allowed the cataleptic animals to come down from the bar within 60 seconds and remained in a catalepsy-free state for at least 60 minutes.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A compound of the following formula: 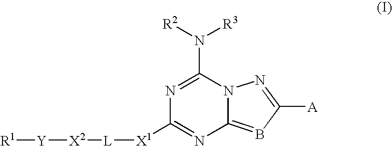
or a pharmaceutically acceptable salt or N-oxide thereof;
wherein
A is aryl or heteroaryl;
B is N or CR²;
each of R² and R³ is independently hydrogen, alkyl, cycloalkyl, cycloalkenyl, aryl, aralkyl, heterocycloalkyl, heterocycloalkenyl, heteroaryl, or heteroaralkyl;
each of X¹ and X² is independently C₁₋₆ alkylene, C₂₋₆ alkenylene, C₂₋₆ alkynylene, or a bond;
L is a bond or a linker selected from the group consisting of: 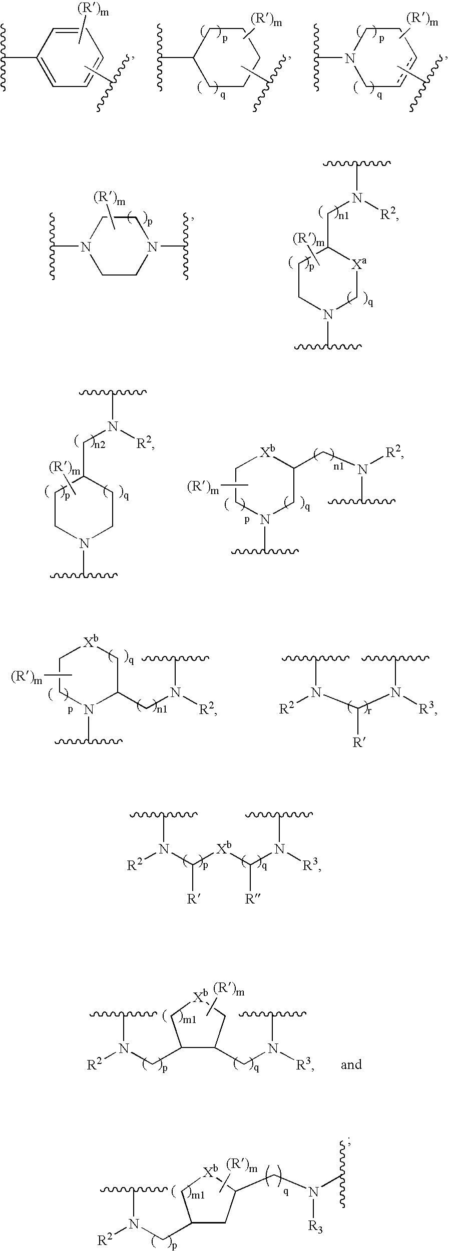
wherein:
each of R′ and R″, independently, is hydrogen, alkyl, alkenyl, alkynyl, alkoxy, acyl, halo, hydroxy, amino, nitro, oxo, thioxo, cyano, guanadino, amidino, carboxy, sulfo, sulfoxy, mercapto, alkylsulfanyl, alkylsulfinyl, alkylsulfonyl, aminocarbonyl, alkylcarbonylamino, alkylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, heterocycloalkyl, heterocycloalkyloxy, heterocycloalkylsulfanyl, aryl, aryloxy, arylsulfanyl, aroyl, heteroaryl, heteroaryloxy, heteroarylsulfanyl, or heteroaroyl; provided that two adjacent R′ groups can join together to form a 4- to 8-membered optionally substituted cyclic moiety;
X^{a} is —C(R²)(R³)—, —S—, —SO—, or —SO₂—;
X^{b} is —C(R²)(R³)—, —NR²—, —O—, —S—, —SO—, or —SO₂—;
each of p, q, m, and m1, is independently 0-3;
r is 1 or 2;
n1 is 0-6; and
n2 is 2-6;
Y is —C(R²)(R³)—, —O—, —S—, —SO—, —SO₂—, —CO—, —CO₂—, or a bond; and
R¹ is alkyl, alkenyl, alkynyl, cycloalkyl, cycloalkylalkyl, cycloalkenyl, cycloalkenylalkyl, aryl, aralkyl, heterocyclyl, or heterocyclylalkyl;
provided that
(1) when L is 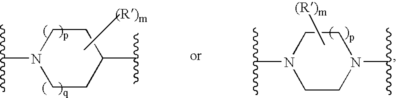
then X¹ is C₁₋₆ alkylene, C₂₋₆ alkenylene, or C₂₋₆ alkynylene;
(2) when L is 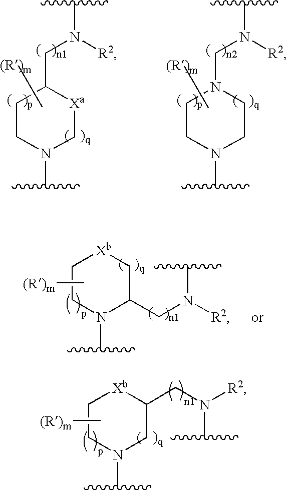
then R¹ is aryl or heteroaryl; and
(3) when L is a bond, X¹ is an alkynylene.

2. The compound of claim 1, wherein X¹ is C₂₋₆ alkynylene.

3. The compound of claim 2, wherein L is 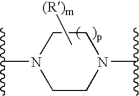
or a bond.

4. The compound of claim 2, wherein X² is C₁₋₄ alkylene or a bond.

5. The compound of claim 2, wherein Y is a bond.

6. The compound of claim 2, wherein each of R² and R³ is independently hydrogen or alkyl.

7. The compound of claim 2, wherein R¹ is alkyl, cycloalkyl, aryl, heterocycloalkyl, or heteroaryl.

8. The compound of claim 7, wherein R¹ is optionally substituted with alkyl, halo, hydroxy, or phenyl.

9. The compound of claim 2, wherein L is 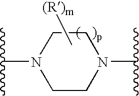
or a bond; X² is C₁₋₄ alkylene or a bond; each of R² and R³ is independently hydrogen or alkyl; R¹ is alkyl, cycloalkyl, aryl, heterocycloalkyl, or heteroaryl, each of which being optionally substituted with alkyl, halo, hydroxy, or phenyl; A is heteroaryl; and B is N.

10. The compound of claim 1, wherein L is

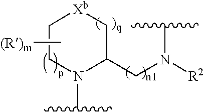

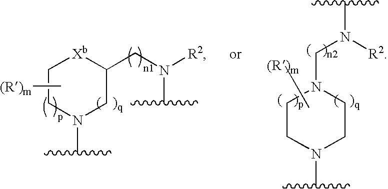

11. The compound of claim 10, wherein X^{b} is —C(R²)(R³)— or —NR²—.

12. The compound of claim 11, wherein X^{b} is —C(R²)(R³)—.

13. The compound of claim 12, wherein p is 0-1 and q is 1.

14. The compound of claim 13, wherein nil is 14 and n2 is 2-4.

15. The compound of claim 14, wherein X¹ is C₁₋₆ alkylene or a bond.

16. The compound of claim 14, wherein X² is C₁₋₆ alkylene or a bond.

17. The compound of claim 14, wherein Y is —SO₂—, —CO—, —CO₂—, or a bond.

18. The compound of claim 14, wherein each of R² and R³ is independently hydrogen or alkyl.

19. The compound of claim 14, wherein R¹ is aryl or heteroaryl, each of which being optionally substituted with alkyl, halo, hydroxy, or phenyl.

20. The compound of claim 14, wherein each of X¹ and X² is independently C₁₋₆ alkylene or a bond; Y is —SO₂—, —CO—, —CO₂—, or a bond; each of R² and R³ is independently hydrogen or alkyl; and R¹ is aryl or heteroaryl, each of which being optionally substituted with alkyl, halo, hydroxy, or phenyl.

21. The compound of claim 14, wherein L is 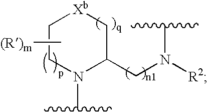
X¹ is a bond; X² is C₁₋₄ alkylene; Y is a bond; each of R² and R³ is independently hydrogen or alkyl; R¹ is aryl or heteroaryl, each of which being optionally substituted with alkyl, halo, hydroxy, or phenyl; A is heteroaryl; and B is N.

22. The compound of claim 1, wherein L is

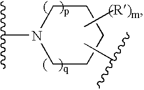

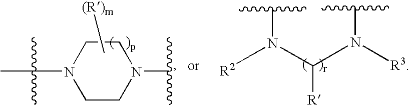

23. The compound of claim 22, wherein X¹ is C₁₋₆ alkylene, C₂₋₆ alkynylene, or a bond.

24. The compound of claim 22, wherein X² is C₁₋₆ alkylene or a bond.

25. The compound of claim 22, wherein Y is SO₂—, —CO—, —CO₂—, or a bond.

26. The compound of claim 22, wherein each of R² and R³ is independently hydrogen or alkyl.

27. The compound of claim 22, wherein R¹ is alkyl, cycloalkyl, aryl, heterocycloalkyl, or heteroaryl.

28. The compound of claim 27, wherein R¹ is optionally substituted with alkyl, halo, hydroxy, or phenyl.

29. The compound of claim 22, wherein X¹ is C₁₋₆ alkylene, C₂₋₆ alkylene, or a bond; X² is C₁₋₆alkylene or a bond; Y is —SO₂—, —CO—, —CO₂—, or a bond; each of R² and R³ is independently hydrogen or alkyl; R¹ is alkyl, cycloalkyl, aryl, heterocycloalkyl, or heteroaryl, each of which being optionally substituted with alkyl, halo, hydroxy, or phenyl; A is heteroaryl; and B is N.

29. The compound of claim 1, said compound being
2-furan-2-yl-N⁷-[1-(5-methyl-isoxazol-3-ylmethyl)-pyrrolidin-2-ylmethyl]-[1,2,4]triazolo[1,5-c]pyrimidine-5,7-diamine;
2-furan-2-yl-N⁷-methyl-N⁷-[1-(5-methyl-isoxazol-3-ylmethyl]-pyrrolidin-2-ylmethyl]-[1,2,4]triazolo[1,5-c]pyrimidine-5,7-diamine;
N⁷-[1-(2-chloro-6-fluoro-benzyl)-pyrrolidin-2-ylmethyl]-2-furan-2-yl-[1,2,4]triazolo[1,5-c]pyrimidine-5,7-diamine;
2-furan-2-yl-N⁷-(1-pyridin-4-ylmethyl-pyrrolidin-2-ylmethyl)-[1,2,4]triazolo[1,5-c]pyrimidine-5,7-diamine;
N⁷-[1-(2,6-dichloro-pyridin-4-ylmethyl)-pyrrolidin-2-ylmethyl]-2-furan-2-yl-[1,2,4]triazolo[1,5-c]pyrimidine-5,7-diamine;
N⁷-[1-(2-chloro-pyridin-4-ylmethyl)-pyrrolidin-2-ylmethyl]-2-furan-2-yl-[1,2,4]triazolo[1,5-c]pyrimidine-5,7-diamine;
N⁷-[1-(2,3-difluoro-benzyl)-pyrrolidin-2-ylmethyl]-2-furan-2-yl-[1,2,4]triazolo[1,5-c]pyrimidine-5,7-diamine;
2-furan-2-yl-N⁷-methyl-N⁷-[1-(2,3,6-trifluoro-benzyl)-piperidin-2-ylmethyl]-[1,2,4]triazolo[1,5-c]pyrimidine-5,7-diamine;
N⁷-[1-(2,4-difluoro-benzyl)-piperidin-2-ylmethyl]-2-furan-N⁷-methyl-[1,2,4]triazolo[1,5-c]pyrimidine-5,7-diamine;
N⁷-[1-(2,6-difluoro-benzyl)-piperidin-2-ylmethyl]-2-furan-2-yl-N⁷-methyl-[1,2,4]triazolo[1,5-c]pyrimidine-5,7-diamine;
N⁷-[1-(2-fluoro-benzyl)-pyrrolidin-2-ylmethyl]-2-furan-2-yl-N⁷-methyl-[1,2,4]triazolo[1,5-c]pyrimidine-5,7-diamine;
N⁷-[1-(5-chloro-furan-2-ylmethyl)-pyrrolidin-2-ylmethyl]-2-furan-2-yl-N⁷-methyl-[1,2,4]triazolo[1,5-c]pyrimidine-5,7-diamine;
N⁷-(1-benzofuran-2-ylmethyl-pyrrolidin-2-ylmethyl)-2-furan-2-yl-N⁷-methyl-[1,2,4]triazolo[1,5-c]pyrimidine-5,7-diamine;
N⁷-[1-(5-chloro-1-methyl-3-trifluoromethyl-1H-pyrazol-4-ylmethyl)-pyrrolidin-2-ylmethyl]-2-furan-2-yl-N⁷-methyl-[1,2,4]triazolo[1,5-c]pyrimidine-5,7-diamine;
N⁷-[1-(2,3-difluoro-benzyl)-pyrrolidin-2-ylmethyl]-2-furan-2-yl-N⁷-methyl-[1,2,4]triazolo[1,5-c]pyrimidine-5,7-diamine;
2-furan-2-yl-N⁷-methyl-N⁷-(1-pyridin-2-ylmethyl-pyrrolidin-2-ylmethyl)-[1,2,4]triazolo[1,5-c]pyrimidine-5,7-diamine;
2-furan-2-yl-N⁷-methyl-N⁷-(1-pyridin-3-ylmethyl-pyrrolidin-2-ylmethyl)-[1,2,4]triazolo[1,5-c]pyrimidine-5,7-diamine;
2-furan-2-yl-N⁷-methyl-N⁷-(1-pyridin-4-pyrrolidin-2-ylmethyl)-[1,2,4]triazolo[1,5-c]pyrimidine-5,7-diamine;
N⁷-[1-(6-chloro-pyridin-3-ylmethyl)-pyrrolidin-2-ylmethyl]-2-furan-2-yl-N⁷-methyl-[1,2,4]triazolo[1,5-c]pyrimidine-5,7-diamine;
2-furan-2-yl-N⁷-methyl-N⁷-[1-(2,3,5,6-tetrafluoro-benzyl)-pyrrolidin-2-ylmethyl]-[1,2,4]triazolo[1,5-c]pyrimidine-5,7-diamine;
1-(5-amino-2-furan-2-yl-[1,2,4]triazolo[1,5-c]pyrimidin-7-ylethynyl)-cyclopentanol;
1-(5-amino-2-furan-2-yl-[1,2,4]triazolo[1,5-c]pyrimidin-7-ylethynyl)-cyclohexanol;
4-(5-amino-2-furan-2-yl-[1,2,4]triazolo[1,5-c]pyrimidin-7-yl)-2-phenyl-but-3-yn-2-ol;
7-(3-cyclohexyl-prop-1-ynyl)-2-furan-2-yl-[1,2,4]triazolo[1,5-c]pyrimidin-5-ylamine;
7-{3-[4-(2,4-difluoro-phenyl)-piperazin-1-yl]-prop-1-ynyl}-2-furan-2-yl-[1,2,4]triazolo[1,5-c]pyrimidin-5-ylamine;
7-{3-[4-(2,4-difluoro-phenyl)-piperazin-1-yl]-propyl}-2-furan-2-yl-[1,2,4]triazolo[1,5-c]pyrimidin-5-ylamine;
N⁷-{2-[4-(2,4-difluoro-phenyl)-piperazin-1-yl]-ethyl}-2-furan-2-yl-[1,2,4]triazolo[1,5-c]pyrimidine-5,7-diamine;
2-furan-2-yl-N⁷-{2-[4-(2,4,6-trifluoro-phenyl)-piperazin-1-yl]-ethyl}-([1,2,4]triazolo[1,5-c]pyrimidine-5,7-diamine;
N⁷-{2-[4-(2-fluoro-phenyl)-piperazin-1-yl]-ethyl}-2-furan-2-yl-[1,2,4]triazolo[1,5-c]pyrimidine-5,7-diamine;
N⁷-{2-[4-(2,5-difluoro-phenyl)-piperazin-1-yl]-ethyl}-2-furan-2-yl-[1,2,4]triazolo[1,5-c]pyrimidine-5,7-diamine;
2-furan-2-yl-N⁷-methyl-N⁷-[2-(4-phenyl-piperazin-1-yl)-ethyl]-[1,2,4]triazolo[1,5-c]pyrimidine-5,7-diamine;
N⁷-{2-[4-(2,4-difluoro-phenyl)-piperazin-1-yl]-ethyl}-2-furan-2-yl-N-7-methyl-[1,2,4]triazolo[1,5-c]pyrimidine-5,7-diamine; and
2-furan-2-yl-N⁷-methyl-N⁷-{2-[4-(2,4,6-trifluoro-phenyl)-piperazin-1-yl]-ethyl}-[1,2,4]triazolo[1,5-c]pyrimidine-5,7-diamine.

30. The compound of claim 1, said compound being
2-furan-2-yl-N⁷-[1-(5-methyl-isoxazol-3-ylmethyl)-pyrrolidin-2-ylmethyl]-[1,2,4]triazolo[1,5-c]pyrimidine-5,7-diamine;
2-furan-2-yl-N⁷-methyl-N⁷-[1-(5-methyl-isoxazol-3-ylmethyl)-pyrrolidin-2-ylmethyl]-[1,2,4]triazolo[1,5-c]pyrimidine-5,7-diamine;
N⁷-[1-(2-chloro-6-fluoro-benzyl)-pyrrolidin-2-ylmethyl]-2-furan-2-yl-[1,2,4]triazolo[1,5-c]pyrimidine-5,7-diamine;
N⁷-[1-(2,6-dichloro-pyridin-4-ylmethyl)-pyrrolidin-2-ylmethyl]-2-furan-2-yl-[1,2,4]triazolo[1,5-c]pyrimidine-5,7-diamine;
N⁷-[1-(5-chloro-furan-2-ylmethyl)-pyrrolidin-2-ylmethyl]-2-furan-2-yl-N⁷-methyl-[1,2,4]triazolo[1,5-c]pyrimidine-5,7-diamine;
N⁷-(1-benzofuran-2-ylmethyl-pyrrolidin-2-ylmethyl)-2-furan-2-yl-N⁷-methyl-[1,2,4]triazolo[1,5-c]pyrimidine-5,7-diamine;
N⁷-[1-(6-chloro-pyridin-3-ylmethyl)-pyrrolidin-2-ylmethyl]-2-furan-2-yl-N⁷-methyl-[1,2,4]triazolo[1,5-c]pyrimidine-5,7-diamine;
2-furan-2-yl-N⁷-methyl-N⁷-[1-(2,3,5,6-tetrafluoro-benzyl)-pyrrolidin-2-ylmethyl]-[1,2,4]triazolo[1,5-c]pyrimidine-5,7-diamine;
1-(5-amino-2-furan-2-yl-[1,2,4]triazolo[1,5-c]pyrimidin-7-ylethynyl)-cyclopentanol;
1-(5-amino-2-furan-2-yl-[1,2,4]triazolo[1,5-c]pyrimidin-7-ylethynyl)-cyclohexanol;
7-(3-cyclohexyl-prop-1-ynyl)-2-furan-2-yl-[1,2,4]triazolo[1,5-c]pyrimidin-5-ylamine;
7-{3-[4-(2,4-difluoro-phenyl)-piperazin-1-yl]-prop-1-ynyl}-2-furan-2-yl-[1,2,4]triazolo[1,5-c]pyrimidin-5-ylamine;
N⁷-{2-[4-(2,4-difluoro-phenyl)-piperazin-1-yl]-ethyl}-2-furan-2-yl-[1,2,4]triazolo[1,5-c]pyrimidine-5,7-diamine;
2-furan-2-yl-N⁷-{2-[4-(2,4,6-trifluoro-phenyl)-piperazin-1-yl]-ethyl}-[1,2,4]triazolo[1,5-c]pyrimidine-5,7-diamine;
N⁷-{2-[4-(2,4-difluoro-phenyl)-piperazin-1-yl]-ethyl}-2-furan-2-yl-N⁷-methyl-[1,2,4]triazolo[1,5-c]pyrimidine-5,7-diamine; and
2-furan-2-yl-N⁷-methyl-N⁷-{2-[4-(2,4,6-trifluoro-phenyl)-piperazin-1-yl]-ethyl}-[1,2,4]triazolo[1,5-c]pyrimidine-5,7-diamine.

31. The compound of claim 1, said compound being
2-furan-2-yl-N⁷-[1-(5-methyl-isoxazol-3-ylmethyl)-pyrrolidin-2-ylmethyl]-[1,2,4]triazolo[1,5-c]pyrimidine-5,7-diamine;
2-furan-2-yl-N⁷-methyl-N⁷-[1-(5-methyl-isoxazol-3-ylmethyl)-pyrrolidin-2-ylmethyl]-[1,2,4]triazolo[1,5-c]pyrimidine-5,7-diamine;
N⁷-[1-(2-chloro-pyridin-4-ylmethyl)-pyrrolidin-2-ylmethyl]-2-furan-2-yl-[1,2,4]triazolo[1,5-c]pyrimidine-5,7-diamine;
N⁷-[1-(5-chloro-furan-2-ylmethyl)-pyrrolidin-2-ylmethyl]-2-furan-2-yl-N⁷-methyl-[1,2,4]triazolo[1,5-c]pyrimidine-5,7-diamine;
N⁷-(1-benzofran-2-ylmethyl-pyrrolidin-2-ylmethyl)-2-furan-2-yl-N⁷-methyl-[1,2,4]triazolo[1,5-c]pyrimidine-5,7-diamine;
7-(3-cyclohexyl-prop-1-ynyl)-2-furan-2-yl-[1,2,4]triazolo[1,5-c]pyrimidin-5-ylamine;
7-{3-[4-(2,4-difluoro-phenyl)-piperazin-1-yl]-prop-1-ynyl}-2-furan-2-yl-[1,2,4]triazolo[1,5-c]pyrimidin-5-ylamine;
N⁷-{2-[4-(2,4-difluoro-phenyl)-piperazin-1-yl]-ethyl}-2-furan-2-yl-[1,2,4]triazolo[1,5-c]pyrimidine-5,7-diamine; and
N⁷-{2-[4-(2,4-difluoro-phenyl)-piperazin-1-yl]-ethyl}-2-furan-2-yl-N⁷-methyl-[1,2,4]triazolo[1,5-c]pyrimidine-5,7-diamine.

32. A pharmaceutical composition comprising a compound of claim 1 and a pharmaceutically acceptable carrier.

33. A pharmaceutical composition comprising a compound of claim 29 and a pharmaceutically acceptable carrier.

34. A method of modulating the A₂ₐ adenosine receptor signaling pathways in a subject, the method comprising administering to said subject with an effective amount of a compound of claim 1.

35. A method of modulating the A₂ₐ adenosine receptor signaling pathways in a subject, the method comprising administering to said subject with an effective amount of a compound of claim 29.

36. A method of inhibiting the A₂ₐ adenosine receptor in a cell, the method comprising the step of contacting said cell with an effective amount of a compound of claim 1.

37. A method of inhibiting the A₂ₐ adenosine receptor in a cell, the method comprising the step of contacting said cell with an effective amount of a compound of claim 29.

38. A method of treating or preventing a disorder or disease in a subject wherein the cause or syndrome of the disorder or disease is associated with an a activation of the A₂ₐ adenosine receptor, the method comprising the step of administering to said subject an effective amount of a compound of claim 1.

39. A method of treating or preventing a disorder or disease in a subject wherein the cause or syndrome of the disorder or disease is associated with an activation of the A₂ₐ adenosine receptor, the method comprising the step of administering to said subject an effective amount of a compound of claim 29.

40. The method of claim 38 or 39, wherein the disorder or disease is selected from the group consisting of Parkinson's disease, pr progressive supranuclear palsy, multiple system atrophy, Alzheimer's disease, depression, AIDS encephalopathy, multiple sclerosis, amyotrophic lateral sclerosis, migraine, attention deficit disorder, narcolepsy, sleep apnea that results in excessive daytime sleepiness, Huntington's disease, cerebral ischemia, brain trauma, hepatic fibrosis, cirrhosis, and fatty liver.

41. The method of claim 40, wherein the disorder or disease is Parkinson's disease.

42. The method of claim 40, wherein the disorder or disease is depression.

43. The method of claim 40, wherein the disorder or disease is migraine.

44. The method of claim 40, wherein the disorder or disease is hepatic fibrosis.

45. The method of claim 40, wherein the disorder or disease is Huntington's disease.
